(19) United States
(12) Patent Application Publication
(10) Pub. No.: US 2023/0285450 A1
(43) Pub. Date: Sep. 14, 2023

(54) PLASMA RADICAL SOLUTION AND METHOD FOR PREPARING SAME

(71) Applicants: PLARIT CO.,LTD., Jeollabuk-do (KR); INNOVATION FOR CREATIVE DEVICES CO., LTD., Anseong-si Gyeonggi-do (KR); AJOU UNIVERSITY INDUSTRY-ACADEMIC COOPERATION FOUNDATION, Suwon-si Gyeonggi-do (KR)

(72) Inventors: Chul Ho KIM, Seoul (KR); Seung Ho LEE, Yongin-si (KR); Young Suk YOU, Pyeongtaek-si (KR); Jin Young HUH, Anseong-si (KR); Myeong Hoon LEE, Suwon-si (KR); Hyo Jung LIM, Gunpo-si (KR)

(73) Assignees: PLARIT CO.,LTD., Jeollabuk-do (KR); INNOVATION FOR CREATIVE DEVICES CO., LTD., Anseong-si Gyeonggi-do (KR); AJOU UNIVERSITY INDUSTRY-ACADEMIC COOPERATION FOUNDATION, Suwon-si Gyeonggi-do (KR)

(21) Appl. No.: 18/318,242

(22) Filed: May 16, 2023

Related U.S. Application Data
(63) Continuation of application No. PCT/KR2021/016564, filed on Nov. 12, 2021.

(30) Foreign Application Priority Data
Nov. 16, 2020 (KR) 10-2020-0153027

(51) Int. Cl. A61K 33/00; A61P 31/04; A61P 17/02; A61P 31/12

(52) U.S. Cl. CPC A61K 33/00; A61P 31/04; A61P 17/02; A61P 31/12

(57) ABSTRACT

The present invention relates to a plasma radical solution and a method for preparing the same, and more particularly, to a plasma radical solution and a method for preparing the same capable of being used in various bio-fields such as sterilization, antibacterial, wound healing, acne relief, atopy treatment, psoriasis treatment, dermatitis treatment, viral skin disease treatment, skin soothing and muscle regeneration by preparing a plasma radical solution containing radicals generated through plasma.

### CROSS-REFERENCE TO RELATED APPLICATIONS

The present application is a Continuation of International Patent Application No. PCT/KR2021/016564 filed on Nov. 12, 2021, which claims priority to and the benefit of Korean Patent Application No. 10-2020-0153027 filed in the Korean Intellectual Property Office on Nov. 16, 2020, the entire contents of which are incorporated herein by reference.

### TECHNICAL FIELD

The present invention relates to a plasma radical solution and a method for preparing the same, and more particularly, to a plasma radical solution and a method for preparing the same capable of being used for various bio-fields such as sterilization, antibacterial, wound healing, acne relief, atopy treatment, psoriasis treatment, dermatitis treatment, viral skin disease treatment, skin soothing and muscle regeneration by preparing a plasma radical solution including radicals generated through plasma.

### BACKGROUND ART

Plasma is called a fourth material state, and refers to a state in which negatively charged electrons, positively charged ions and neutral particles (molecules and atoms) are mixed. Electrons may be easily accelerated by various methods, and neutral particles collide with molecules to generate chemically active species, and the ions create conditions in which chemical reactions may occur on the surface of an object to be treated, so that active species may cause active chemical reactions on the surface. In addition, since plasma may be generated from high vacuum even to high pressures above atmospheric pressure, and can be discharged not only in a gas state but also in a liquid state, there is an advantage of being able to selectively use a plasma generation method suitable for various production conditions, and thus, the plasma has been used in various industrial fields such as semiconductors, displays, energy, machinery, chemistry and bio. In particular, the plasma may have excellent sterilization power through the generation of active species having strong oxidizing power and high reactivity, and thus has been actively used in bio-fields such as life/medical fields, food industry, and agriculture. Recently, studies have been conducted to fundamentally treat incurable diseases such as atopy, skin disease, Alzheimer's disease, Parkinson's disease, and cancer, which are diseases related to proteins using active species.

A plasma radical solution is a solution in which various active species are dissolved in water by reacting active species including ions and radicals generated from plasma with water. In particular, when active species generated through plasma are dissolved in distilled water, conductivity increases, and various reactive oxygen species (ROS) and reactive nitrogen species (RNS) are generated, so that pH, and oxidation/reduction potential (ORP) values are changed. Among these, the RNS include NO₂— (nitrate), NO₃-(nitrite), ONOO— (peroxynitrite), etc., and may be used for sterilization and antibacterial for bacteria, fungi, and viruses. In particular, an ONOO— compound called peroxynitrite has been applied to various bio-fields such as wound healing, promotion of cell activity, and treatment of skin diseases, but has a problem that it is difficult to prepare solutions containing peroxynitrite. This is because peroxynitrite is generated in the solution by unstable radicals generated by plasma and thus is difficult to be naturally synthesized. In addition, there has been a problem of existing technologies that since other radicals are unstable, the radicals change into other substances when dissolved in a solution to lose their properties in a short time (life time: several nanoseconds). However, in the case of the plasma radical solution prepared in the present invention, the generation and decomposition of peroxynitrite continuously occur in the solution, and thus, there is an advantage in maintaining bio-properties for a long time.

At this time, the plasma radical solution has sterilization and antibacterial effects under conditions of constant pH and NO₃— concentration. Therefore, there is a need for a technique for maintaining the sterilization and antibacterial effects through a method of adjusting the pH and NO₃— concentration of the plasma radical solution. In the case of the present invention, there is an advantage in maintaining the sterilization and antibacterial effects of the plasma radical solution by constantly adjusting the pH of the plasma radical solution using NaH₂PO₄.

### DETAILED DESCRIPTION OF THE INVENTION

### Technical Problem

The present invention is derived to prepare a solution containing peroxynitrite harmless to the human body and apply the prepared solution to bio-fields such as sterilization, antibacterial and skin treatment. An object of the present invention is to provide a plasma radical solution with bio-properties and a method for preparing the same capable of preparing a solution containing peroxynitrite by generating plasma and using the prepared solution for sterilization and antibacterial against bacteria, fungi and viruses, skin wound healing, acne relief, atopy treatment, psoriasis treatment, dermatitis treatment, viral skin disease treatment, skin soothing, muscle regeneration, and the like.

Another object of the present invention is to provide a plasma radical solution and a method for preparing the same capable of constantly maintaining the pH and NO₃-concentration.

Yet another object of the present invention is to provide a plasma radical solution with non-toxic properties that are harmless to the human body and a method for preparing the same.

### Technical Solution

According to an embodiment of the present invention, a plasma radical solution includes active species that sterilize any one or more of bacteria, fungi and viruses; and a solvent in which the active species are dissolved, wherein the active species are generated through plasma.

The active species may include at least one of reactive nitrogen species and reactive oxygen species, and the reactive nitrogen species may include peroxynitrite (ONOO—).

The plasma radical solution may sterilize any one or more of bacteria, fungi and viruses at pH 0 to 4.

The plasma radical solution may sterilize any one or more of bacteria, fungi and viruses at a NO₃— (nitrite) concentration of 400 ppm or higher.

The plasma radical solution may sterilize any one or more of bacteria, fungi and viruses at conductivity of 1000 μs/cm or higher.

The plasma radical solution may be stored at a temperature of 85° C. or less.

The plasma radical solution may have an expiration period of 6 months or more.

The plasma radical solution may have a sterilization effect on the bacteria including any one or more of gram positive bacteria and gram negative bacteria, wherein the gram positive bacteria may include any one or more of Staphylococcus aureus, Bacillus cereus, Bacillus subtilis, Enterococcus, Streptococcus, and Lactococcus, and the gram negative bacteria may include any one or more of Escherichia coli, Salmonella typhimurium, Pseudomonas aeruginosa, Klebsiella penumoniae, Haemophilus influenzae, Enterobacter aerogenes and Yersinia pestis.

The plasma radical solution may have a sterilization effect on the fungi including any one or more of Candida and Aspergillus.

The plasma radical solution may have a sterilization effect on the viruses including influenza virus.

The plasma radical solution may have bio properties that are able to be used for antibacterial, skin wound healing, acne relief, atopy treatment, psoriasis treatment, dermatitis treatment, viral skin disease treatment, skin soothing and muscle regeneration.

The solvent may be selected by any one of distilled water and purified water.

According to another embodiment of the present invention, a method for preparing a plasma radical solution includes (a) generating active species including nitrogen oxides by generating plasma; (b) oxidizing the active species using at least one of a corona discharge and a catalyst; (c) preparing a plasma radical solution by dissolving the oxidized active species in a solvent; and (d) adjusting the pH of the plasma radical solution.

In the step (c), the oxidized active species may be dissolved in the solvent through bubbling, and the plasma radical solution may include peroxynitrite (ONOO—).

In step (d), the pH of the plasma radical solution may be adjusted to 0 to 4.

### Advantageous Effects

According to the present invention, it is possible to provide a plasma radical solution with bio-properties and a method for preparing the same capable of preparing a solution containing peroxynitrite by generating plasma and using the prepared solution for sterilization and antibacterial against bacteria, fungi and viruses, skin wound healing, acne relief, atopy treatment, psoriasis treatment, dermatitis treatment, viral skin disease treatment, skin soothing, muscle regeneration, and the like.

Further, it is possible to provide a plasma radical solution and a method for preparing the same capable of constantly maintaining the pH and NO₃— concentration.

Further, it is possible to provide a plasma radical solution with non-toxic properties that are harmless to the human body and a method for preparing the same.

### BRIEF DESCRIPTION OF DRAWINGS

FIG. 1A is a graph showing changes in pH of a plasma radical solution according to a plasma treatment time according to an embodiment of the present invention; FIG. 1B is a graph showing changes in NO₃— (nitrite) concentration of a plasma radical solution according to an embodiment of the present invention; and FIG. 1C is a graph showing changes in conductivity of the plasma radical solution according to an embodiment of the present invention.

FIG. 2 is a diagram illustrating a sterilization effect according to a storage temperature when the plasma radical solution is stored.

FIG. 3 is a graph showing changes in NO₃— (nitrite) concentration and changes in antibacterial and sterilization effects when a plasma radical solution is prepared and then stored at room temperature for 6 months or more.

FIG. 4 is a diagram illustrating results of sterilization tests for E. coli, S. aureus, S. typhimurium, P. aeruginosa and B. cereus of the plasma radical solution according to the present invention.

FIG. 5 is a diagram illustrating results of sterilization tests for S. typhimurium and S. aureus according to a pH of the plasma radical solution.

FIG. 6 is a diagram illustrating antibacterial and sterilization effective concentration tests for S. aureus, E. coli and S. typhimurium according to a NO₃— (nitrite) concentration of the plasma radical solution.

FIG. 7 is a diagram illustrating test results for confirming sterilizing active ingredients of a plasma radical solution using a peroxynitrite (ONOO—) scavenger.

FIG. 8 is a diagram illustrating test results for confirming sterilizing active ingredients of a plasma radical solution by comparing different nitrogen oxides without using plasma.

FIG. 9 is a diagram illustrating results of a sterilization test for viruses of a plasma radical solution.

FIG. 10 is a diagram illustrating results of a skin wound healing effect test of a plasma radical solution.

FIG. 11 is a diagram illustrating results of a sterilization test for acne bacteria of a plasma radical solution.

FIG. 12 is a graph showing evaluation results of XTT and CCK8 toxicity of a plasma radical solution.

FIG. 13 is a diagram illustrating evaluation results of Annexin V/PI toxicity of a plasma radical solution.

FIG. 14 is a diagram illustrating evaluation results of Live and dead assay of a plasma radical solution.

FIG. 15 is a diagram illustrating evaluation results of bovine corneal opacity and permeability (BCOP) assay of a plasma radical solution.

FIG. 16 is a graph showing evaluation results of a skin irritation test of a plasma radical solution.

FIG. 17 is a graph showing evaluation results of an acute oral toxicity test of a plasma radical solution.

FIG. 18 is a graph showing evaluation results of a phototoxicity test of a plasma radical solution.

FIG. 19 is a flowchart of a method for preparing a plasma radical solution according to the present invention.

FIG. 20 is a graph showing the generation amounts of nitrogen oxides according to plasma power.

### BEST MODE

The present invention will be described below in detail with reference to the accompanying drawings. Herein, the repeated description and the detailed description of publicly-known function and configuration that may make the gist of the present invention unnecessarily ambiguous will be omitted. The embodiment of the present invention is provided to more completely describe the present invention to those skilled in the art. Accordingly, shapes, sizes, and the like of elements in the drawings may be exaggerated for clearer explanation.

Throughout this specification, unless explicitly described to the contrary, a case where any part “includes” any component will be understood to imply the inclusion of stated components but not the exclusion of any other component.

Hereinafter, preferred embodiments will be proposed in order to help in understanding of the present invention. However, the following embodiments are just provided to more easily understand the present invention and contents of the present invention are not limited by the embodiments.

<Plasma Radical Solution>

A plasma radical solution according to the present invention may include active species and a solvent.

The active species according to the present invention may be generated through plasma. At this time, a plasma generating device can be used without limitation as long as the plasma generating device may perform plasma discharge, but preferably uses thermal plasma. The plasma may be generated by any one of a dielectric barrier discharge (DBD), corona discharge, arc discharge, microwaves, radio frequency (RF), low frequency (LF) and high frequency (HF) to generate active species.

The active species generated through plasma include at least one of reactive nitrogen species (RNS) and reactive oxygen species (ROS), and may sterilize any one or more of bacteria, fungi, and viruses. The ROS may include O₂— (superoxide anion), H₂O₂ (hydrogen peroxide), OH (hydroxy radical), OH— (hydroxyl anion), O₃ (ozone), and the like. The RNS may include NO₂— (nitrate), NO₃-(nitrite), NO· (nitric oxide radical), NO₂· (nitrogen dioxide radical), ONOO— (peroxynitrite), and the like. In particular, peroxynitrite (ONOO—) is included to have an excellent sterilization effect on any one or more of bacteria, fungi, and viruses.

The solvent according to the present invention may dissolve active species generated by plasma. As the solvent, any material capable of dissolving the active species can be used without limitation, but preferably, any one of distilled water and purified water may be selected.

The plasma radical solution according to the present invention is a solution in which the active species are dissolved in a solvent, and may sterilize any one or more of bacteria, fungi, and viruses, and may have bio-properties that can be used for skin wound healing, acne relief, and atopy treatment.

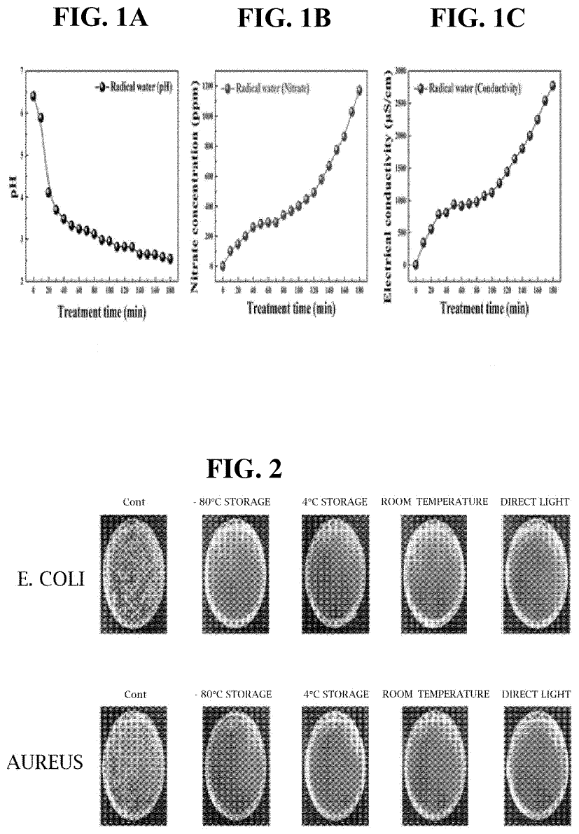

The plasma radical solution may have bio-properties when the pH is 0 to 4, the NO₃-(nitrite) concentration is 400 ppm or more, and the conductivity is 1000 μs/cm or more. FIG. 1A is a graph showing changes in pH of a plasma radical solution according to a plasma treatment time according to an embodiment of the present invention; FIG. 1B is a graph showing changes in NO₃— (nitrite) concentration of a plasma radical solution according to an embodiment of the present invention; and FIG. 1C is a graph showing changes in conductivity of the plasma radical solution according to an embodiment of the present invention. Referring to FIGS. 1A to 1C, these can be seen that the plasma radical solution has a pH of 0 to 4, a concentration of 400 ppm or more, and conductivity of 1000 μs/cm or more within 3 hours after the active species are dissolved in the solvent. The plasma radical solution may have a sterilization effect at a pH of 0 to 4, preferably a sterilization effect at a pH of 1 to 4. If the pH of the plasma radical solution exceeds 4, a problem in which the sterilization effect is reduced may occur. In addition, the sterilization effect may be reduced even when the NO₃— (nitrite) concentration of the plasma radical solution is less than 400 ppm or the conductivity is less than 1000 μs/cm.

In addition, the plasma radical solution may be stored at a temperature of 85° C. or lower, preferably −80° C. to 60° C. When the storage temperature of the plasma radical solution exceeds 85° C., the plasma radical solution may be deteriorated to reduce the sterilization effect. FIG. 2 is a diagram illustrating a sterilization effect according to a storage temperature when the plasma radical solution is stored. Referring to FIG. 2, the plasma radical solution was stored under conditions of −80° C. to 4° C., room temperature and direct light, respectively, and was used to sterilize E. coli and S. aureus. At this time, the sterilization effect on E. coli and S. aureus may be confirmed in the plasma radical solution under all temperature conditions. Accordingly, it can be confirmed that the sterilization effect is maintained even when the plasma radical solution is stored at a temperature of 85° C. or less.

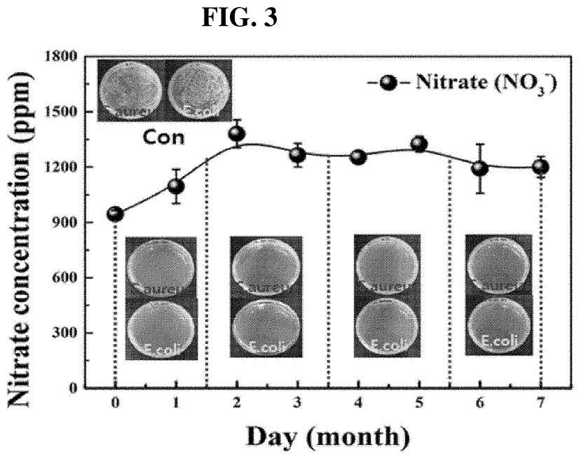

In addition, the plasma radical solution may have an expiration period of 6 months or more. FIG. 3 is a graph showing changes in NO₃— (nitrite) concentration and changes in antibacterial and sterilization effects when a plasma radical solution is prepared and then stored at room temperature for 6 months or more. Referring to FIG. 3, the concentration of NO₃— (nitrite) did not change significantly even after 7 months after the preparation of the plasma radical solution, and the sterilization effect on S. aureus and E. coli was continuously confirmed.

The plasma radical solution may have sterilization and antibacterial effects on various bacteria including gram positive bacteria and gram negative bacteria. The gram positive bacteria may be divided into gram-positive cocci and gram-positive bacilli. The gram-positive cocci may include Staphylococcus aureus, Staphylococcus epidermidis, Streptococcus pneumoniae, Streptococcus pyogenes, and Enterococcus, etc., and the gram-positive bacilli may include Bacillus anthracis, Clostridium difficile, Clostridium tetani, Clostridium botulinum, Listeria monocytogenes, Corynebacterium diphtheria, Mycobacterium tuberculosis, and the like. In addition, the plasma radical solution may have a sterilization effect even on other gram-positive bacteria not disclosed above. Preferably, the plasma radical solution may have a sterilization effect on at least one of Staphylococcus aureus, Bacillus cereus, Bacillus subtilis, Enterococcus, Streptococcus, Lactococcus, etc. The gram negative bacteria may be divided into gram-positive cocci and gram-positive bacilli. The gram-negative cocci may include Meisseria gonorrhoeae, Meisseria meningitides, and the like, and the gram-negative bacilli may include Escherichia coli, Klebsiella penumoniae, Acinetobacter baumannii, Enterobacter aerogenes, Vibrio cholera, Vibrio vulnificus, Pseudomonas aeruginosa, Bacteroides fragilis, etc. In addition, the plasma radical solution may have a sterilization effect even on other gram negative bacteria not disclosed above. Preferably, the plasma radical solution may have an excellent sterilization effect on at least one of Escherichia coli, Salmonella typhimurium, Pseudomonas aeruginosa, Klebsiella penumoniae, Haemophilus influenzae, Enterobacter aerogenes, and Yersinia pestis. In addition, the plasma radical solution may have a sterilization effect on other bacteria such as Mycobacterium tuberculosis, Rickettsia, Chlamydia, Spirochaetales, and Acid fast bacteria, which are not gram-stained.

The plasma radical solution may have sterilization and antibacterial effects on various fungi consisting of Chytridiomycota, Zygomycota, Ascomycota, Basidiomycota, and Deuteromycota. Preferably, the plasma radical solution may have an excellent sterilization effect on Candida and Aspergillus.

The plasma radical solution has sterilization and antibacterial effects on various viruses, and preferably has an excellent sterilization effect on influenza viruses.

The plasma radical solution has bio-properties and may be used as a material that may heal skin wounds. In addition, the plasma radical solution has sterilization and antibacterial effects on acne bacteria and atopic bacteria, and may be used for acne relief and atopic treatment. In addition, the plasma radical solution may alleviate other skin-related diseases such as psoriasis, dermatitis and viral dermatosis. In addition, the plasma radical solution may also be used for skin soothing and muscle regeneration.

### Examples

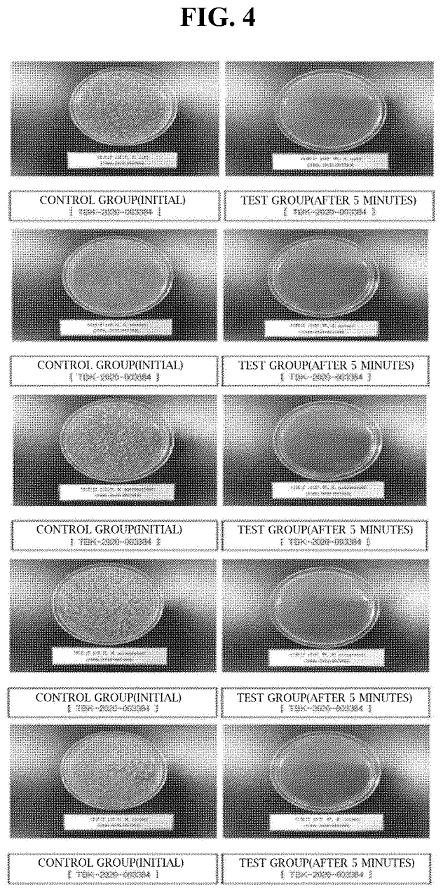

FIG. 4 is a diagram illustrating results of sterilization tests for E. coli, S. aureus, S. typhimurium, P. aeruginosa and B. cereus of a plasma radical solution according to the present invention. Referring to FIG. 4, it can be confirmed that the 5 types of bacteria were sterilized after 5 minutes, and the sterilization test for the 5 types of bacteria was performed as follows.

First, the 5 types of test strains, E. coli, S. aureus, S. typhimurium, P. aeruginosa, and B. cereus, were inoculated using a Tryptic soy broth (TSB) and incubated for 18 hours to 24 hours at a temperature of 34° C. to 36° C.

Second, the mixture was diluted in sterile physiological saline to be the viable cell count of 1.0*10⁸ CFU/ml to 9.9*10⁸ CFU/ml, and used as a test strain solution.

Third, 0.2 ml of the test strain solution was added to 20 ml of the plasma radical solution, mixed, and then left at 20° C. to 24° C. for 5 minutes.

Fourth, the test solution neutralized by first dilution in a D/E neutralizing broth was diluted step by step, and 1 ml of each concentration was dispensed into 2 Petri dishes.

Fifth, 15 ml to 25 ml of previously prepared TSA at 45° C. to 50° C. was dispensed into a petri dish and coagulated at room temperature.

Sixth, the solidified Petri dish was turned over and incubated for 24 to 48 hours at a temperature of 34° C. to 36° C.

Seventh, the viable cell count after culture was observed by selecting a Petri dish representing 30 to 300 cells, and if the viable cell count was observed only at the lowest dilution step, the viable cells were counted regardless of an observation range. When bacteria were proliferated, the viable cell count was calculated by multiplying the cell count on the medium by a dilution rate, and when the bacteria did not grow in the medium, the viable cell count was multiplied by a dilution rate made in the neutralization step and represented as less than 10 (<10). The viable cell count was measured according to [Equation 1], and a Log reduction (LR) value for the sterilization effect was calculated according to [Equation 2]. At this time, the initial and control viable cell counts were measured using sterile physiological saline.

N=C×D (however, satisfied N=viable cell count,C=colony count (average), and D=dilution rate) [Equation 1]

LR=log A−log B (however, satisfied A=log value of viable cell count of control group and B=log value of viable cell count of test group) [Equation 2]

TABLE 1

| | | Viable cell | | |
|---|---|---|---|---|
| | | count | | |
| Test strain | Classification | (CFU/ml) | Log value | LR |
| E. coli | Control group | 4.4 × 10⁶ | 6.64 | >5.64 |
| | (initial) | | | |
| | Test group | <10 | <1.00 | |
| | (after 5 minutes) | | | |
| S. aureus | Control group | 9.4 × 10⁶ | 6.97 | 3.90 |
| | (initial) | | | |
| | Test group | 1.2 × 10³ | 3.07 | |
| | (after 5 minutes) | | | |
| S. typhimurium | Control group | 2.1 × 10⁶ | 6.33 | >5.33 |
| | (initial) | | | |
| | Test group | <10 | <1.00 | |
| | (after 5 minutes) | | | |
| P. aeruginosa | Control group | 8.2 × 10⁶ | 6.91 | >5.91 |
| | (initial) | | | |
| | Test group | <10 | <1.00 | |
| | (after 5 minutes) | | | |
| B. cereus | Control group | 4.7 × 10⁶ | 6.67 | >5.67 |
| | (initial) | | | |
| | Test group | <10 | <1.00 | |
| | (after 5 minutes) | | | |

As a result of the sterilization test for 5 types of bacteria, the LR values of the test strains were >5.64 for E. coli, 3.90 for S. aureus, >5.33 for S. typhimurium, >5.91 for P. aeruginosa and >5.67 for B. cereus, and confirmed that the sterilization effect was 99.9% or higher.

FIG. 5 is a diagram illustrating results of sterilization tests for S. typhimurium and S. aureus according to a pH of a plasma radical solution. Referring to FIG. 5, it can be confirmed that the sterilization effect was exhibited when the pH of the plasma radical solution was adjusted to 3 by adding NaH₂PO₄, and the sterilization test was performed as follows.

First, S. typhimurium and S. aureus were inoculated into a liquid medium and incubated at 37° C. for 18 hours, and the incubated strains were dispensed onto the prepared solid medium according to each dilution ratio.

Second, each sample was dispensed and incubated at 37° C. for 18 hours and diluted to be the viable cell count of 1.0*10⁵ CFU/ml and used as a test strain solution.

Third, 1 ml of the diluted strains were added to a plasma radical solution adjusted to pH 3 using NaOH or NaH₂PO₄, mixed, and left at room temperature for 5 minutes. At this time, after washed with sterilized water (DW) as a control, the same amount of bacteria was dispensed onto the prepared solid medium.

As a result of the test, when NaOH or NaH₂PO₄ was used to adjust the pH of the plasma radical solution, it was confirmed that the sterilization effect of the plasma radical solution disappeared when NaOH was used. On the other hand, it can be seen that the sterilization effect was maintained in the solution of which the pH is adjusted using NaH₂PO₄. Therefore, it can be confirmed that NaH₂PO₄ needs to be used when adjusting the pH of the plasma radical solution.

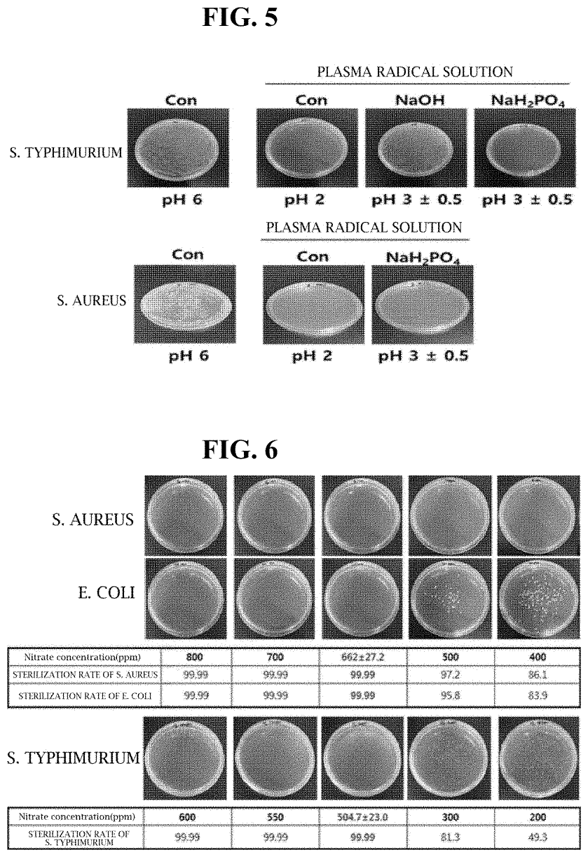

FIG. 6 is a diagram illustrating antibacterial and sterilization effective concentration tests for S. aureus, E. coli and S. typhimurium according to a NO₃— (nitrite) concentration of a plasma radical solution. Referring to FIG. 6, it can be seen that the sterilization effect changes according to a concentration of nitrate, which is a compound that performs sterilization. S. aureus and E. coli showed a 99.99% sterilization effect at a concentration of 662±27.2 ppm or more, and S. typhimurium had a 99.99% sterilization effect at a concentration of 504.7±23.0 ppm or more.

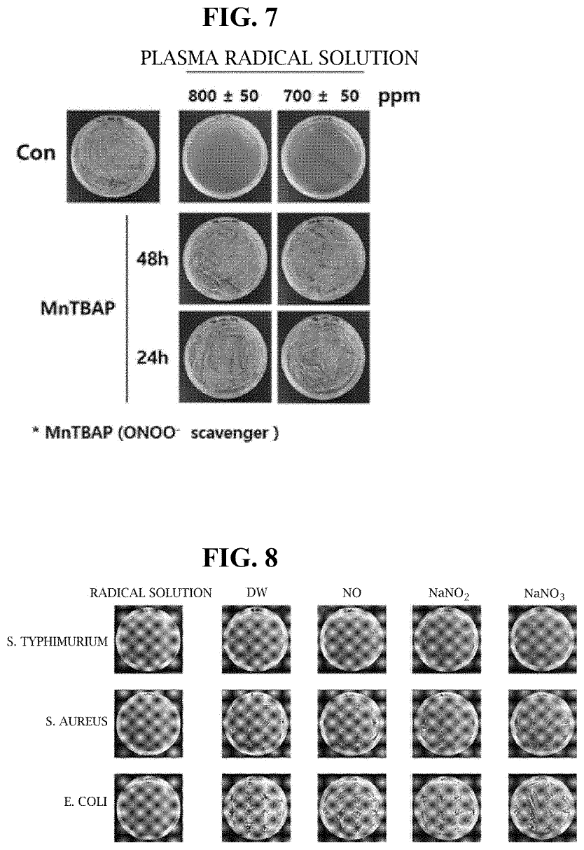

FIG. 7 is a diagram illustrating test results for confirming sterilizing active ingredients of a plasma radical solution using a peroxynitrite (ONOO—) scavenger. In order to confirm a main sterilization ingredient of the plasma radical solution, as a result of using MnTBAP as a scavenger of ONOO—, which was another form of nitrate, it was confirmed that the sterilization effect of the plasma radical solution disappeared. Therefore, it can be confirmed that the main sterilization ingredient of the plasma radical solution is ONOO—.

FIG. 8 is a diagram illustrating test results for confirming sterilizing active ingredients of a plasma radical solution by comparing different nitrogen oxides without using plasma. In order to confirm a sterilization effect of nitrogen oxides without plasma, ionized NO, NO₂ and NO₃ solutions were prepared and then sterilization experiments of S. typhimurium, S. aureus and E. coli were tested. As a result, no sterilization effect was observed in the NO, NO₂ and NO₃ solutions. It can be confirmed that the sterilization effect is not exhibited in the case of nitrogen oxides without plasma.

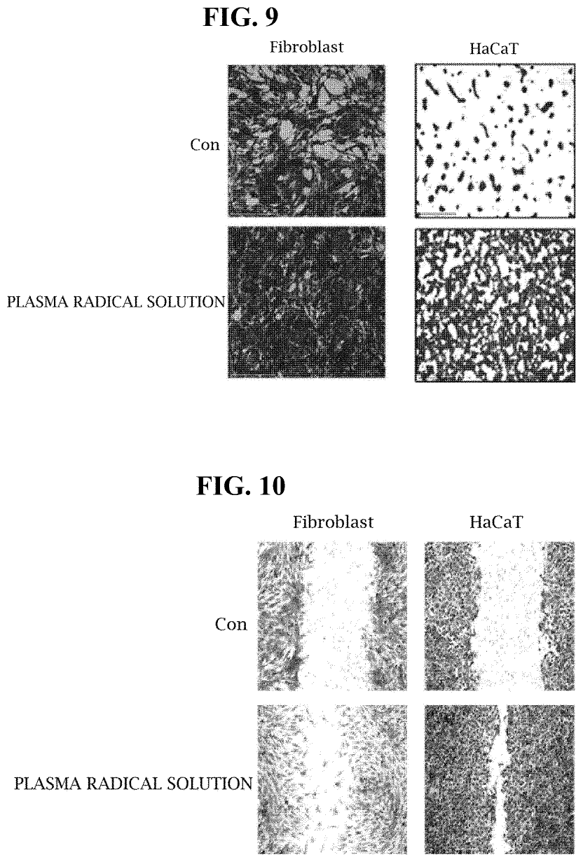

FIG. 9 is a diagram illustrating results of sterilization tests against viruses of a plasma radical solution. First, fibroblasts and normal epidermal cells of HaCaT cells were uniformly cultured, and each cell was supplied with PR8 influenza virus to infect the cells. Then, a mixture of the culture medium and the plasma radical solution, a control medium and a PBS mixture were supplied to infected cells. As a result, it can be confirmed that the death of the cells supplied with the plasma radical solution is suppressed. Therefore, it can be confirmed that the plasma radical solution has an effect of the growth inhibition and sterilization of viruses.

FIG. 10 is a diagram illustrating results of a skin wound healing effect test of the plasma radical solution. First, fibroblasts and normal epidermal cells of HaCaT cells were uniformly cultured, and each cell was wound in the same size. Then, a mixture of the culture medium and the plasma radical solution, a control medium and a PBS mixture were supplied to injured cells. As a result, it can be seen that the wounds were not healed in the cells of the control group, whereas the wounds were healed in the cells supplied with the plasma radical solution. Therefore, it can be confirmed that the plasma radical solution is effective in skin wound healing.

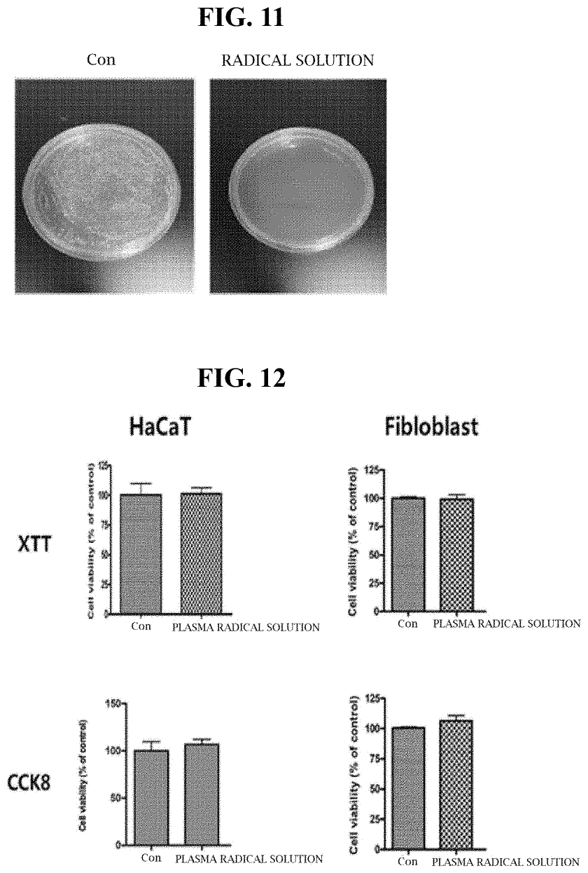

FIG. 11 is a diagram illustrating results of a sterilization test for acne bacteria of a plasma radical solution. Referring to FIG. 10, as a result of a sterilization test for P. acnes which was a main bacterium of acne bacteria, it can be confirmed that the acne bacteria are sterilized when the plasma radical solution is supplied to the acne bacteria. Therefore, it can be confirmed that the plasma radical solution is effective in alleviating acne.

The following are the evaluation results of toxicity of the plasma radical solution according to the present invention.

FIG. 12 is a graph showing evaluation results of XTT and CCK8 toxicity of a plasma radical solution. As a result of XTT assay and CCK8 assay to confirm the toxicity of the plasma radical solution in fibroblasts and HaCaT, all colors of the culture solution did not change when confirmed with naked eye. Referring to FIG. 12, as a result of quantifying the XTT and CCK8 test results through an ELISA reader device, it can be confirmed that the plasma radical solution is non-toxic.

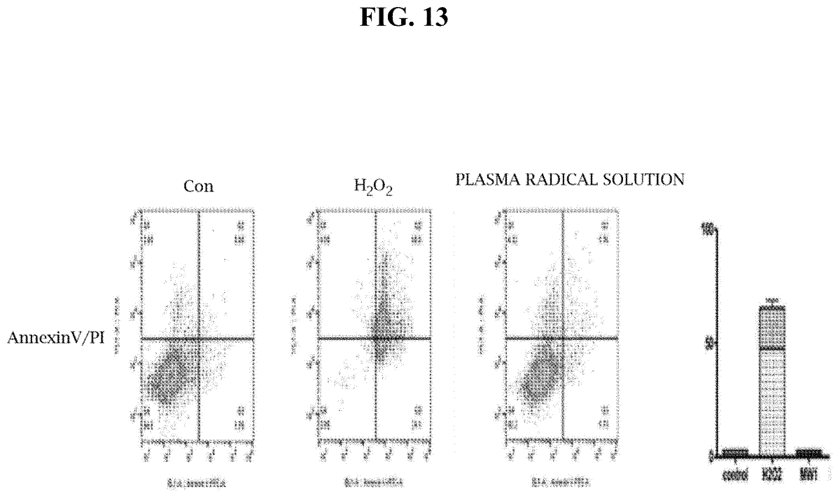

FIG. 13 is a diagram illustrating evaluation results of Annexin V/PI toxicity of a plasma radical solution. In the evaluation of Annexin V/PI toxicity, many spots were present in the upper right end when cells died. Referring to FIG. 13, when H₂O₂ was treated, many spots were present in the upper right end, but in the case of the plasma radical solution, it was confirmed that there was no significant difference between the spots and normal cells, and thus, there was no toxicity.

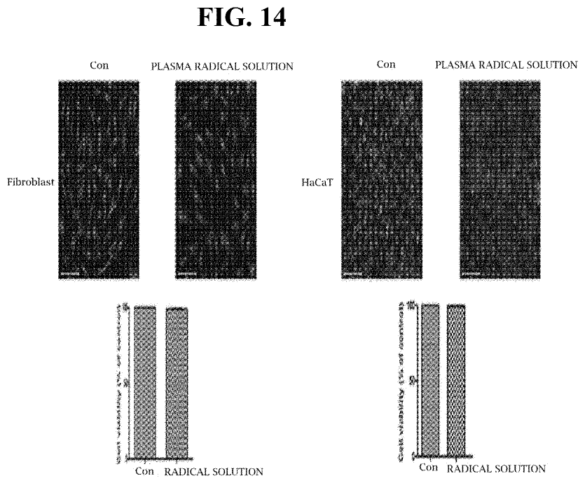

FIG. 14 is a diagram illustrating evaluation results of Live and dead assay of a plasma radical solution. Live and dead assay evaluation was stained green if cells were alive. Referring to FIG. 14, in the case of the plasma radical solution, it can be confirmed that there is no toxicity because there is no significant difference between normal cells and the stained area.

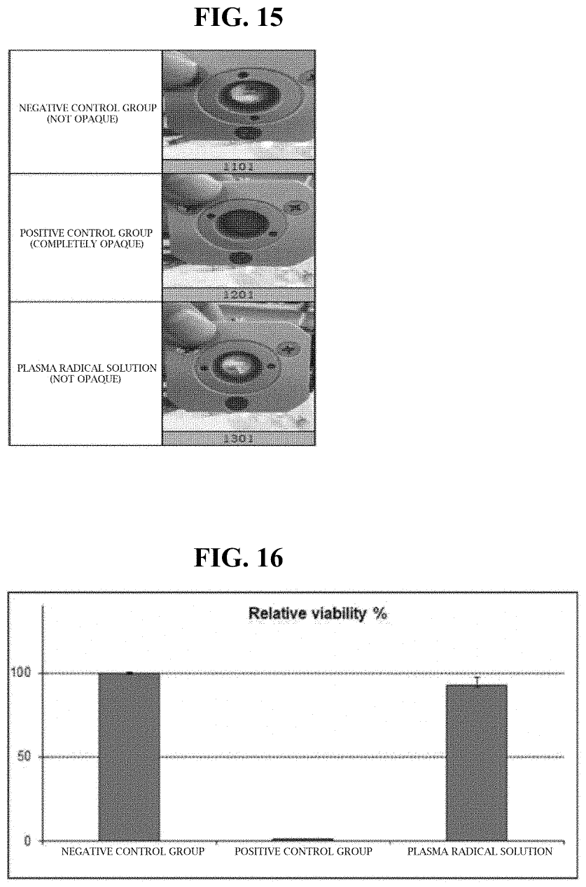

FIG. 15 is a diagram illustrating evaluation results of bovine corneal opacity and permeability (BCOP) assay of a plasma radical solution. The BCOP assay is a test to evaluate an irritation index for the eye mucosa using opacity and permeability. Referring to FIG. 15, it can be seen that a positive control group (ethyl alcohol) is completely opaque, whereas the plasma radical solution is not opaque as in a negative control group (water). An in vitro irritancy score (IVIS) may be calculated by measuring the opacity and permeability, and the IVIS value may be calculated by [Equation 3].

IVIS=Mean opacity unit=(1.5×mean transmitance) [Equation 3]

TABLE 2

| | | Transmittance | |
|---|---|---|---|
| | | (Absorbance | |
| Test group | Opacity unit | (OD₄₉₀)) | IVIS |
| Negative control group: | −0.5 ± 0.7 | 0.003 ± 0.001 | −0.5 ± 0.7 |
| Positive control group | 19.9 ± 0.6 | 1.300 ± 0.203 | 39.4 ± 3.6 |
| Plasma radical solution | 0.7 ± 2.4 | 0.007 ± 0.004 | 0.8 ± 2.5 |

The IVIS value of the plasma radical solution was measured to 3 or less, so that it can be confirmed that there is no irritation to eye mucosa.

FIG. 16 is a graph showing evaluation results of a skin irritation test of the plasma radical solution. In the skin irritation test, the absorbance was measured for a SKINETHIC™ human skin model, and a cell viability value (%) was calculated using [Equation 4] to [Equation 6].

Cell viability (%) of negative control group=[absorbance of negative control group/absorbance of negative control group]×100 [Equation 4]

Cell viability (%) of positive control group=[absorbance of positive control group/absorbance of negative control group]×100 [Equation 5]

Cell viability (%) of plasma radical solution=[absorbance of plasma radical solution/absorbance of negative control group]×100 [Equation 6]

Referring to FIG. 16, a positive control group (sodium dodecyl sulfate, SDS) had a cell viability of about 1.1%, whereas the plasma radical solution had about 92.7±4.7%, similar to a negative control group (phosphate buffer saline, PBS). Since the cell viability exceeds 50% according to a globally harmonized system (GHS) of classification and labeling of chemicals classification, it can be confirmed that the plasma radical solution is a skin non-irritating substance.

The following are evaluation results of a human skin primary irritation test of the plasma radical solution. For the primary stimulation test of the human skin, the plasma radical solution was applied to the test site for 24 hours and the skin reaction was observed to measure the skin reactivity and skin irritation. As a result, it may be confirmed that the skin reactivity was 0.00 and the skin irritation was non-irritating, so that the plasma radical solution is a substance without skin irritation.

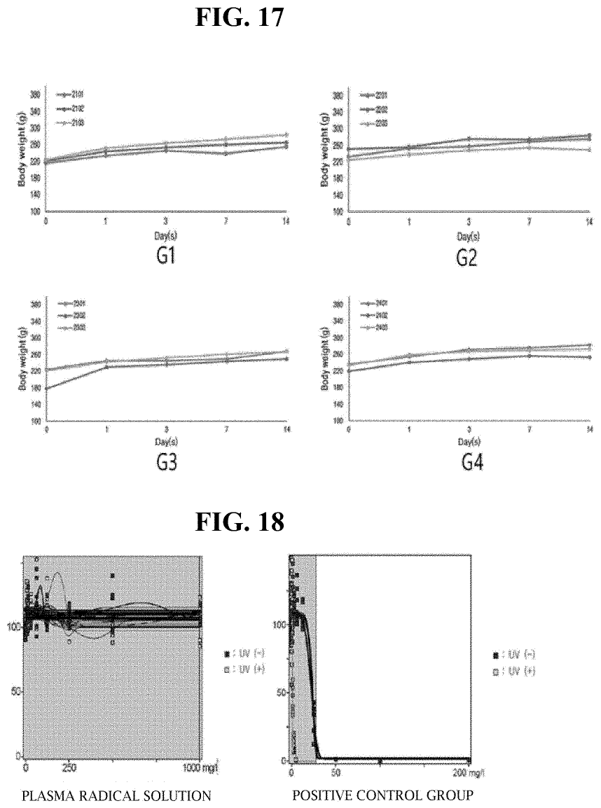

FIG. 17 is a graph showing evaluation results of an acute oral toxicity test of the plasma radical solution. An acute oral toxicity test is a test to evaluate adverse effects in a short time after administering a test substance once or several times within 24 hours, and a plasma radical solution was orally administered to each of three SD-based female rats at 300 mg/kg body weight (B.W.) once for Groups 1 and 2, and at 2000 mg/kg B.W. once for Groups 3 and 4.

TABLE 3

| | | | | Dose | Dosage | |
|---|---|---|---|---|---|---|
| | | Animal | | (mg/kg | (mg/kg | Route of |
| Group | Sex | No. | Number | B.W.) | B.W.) | administration |
| G1 | Female | 2101 | 3 | 300 | 10 | Oral |
| | | to | | | | |
| | | 2103 | | | | |
| G2 | Female | 2201 | 3 | 300 | 10 | Oral |
| | | to | | | | |
| | | 2203 | | | | |
| G3 | Female | 2301 | 3 | 2000 | 10 | Oral |
| | | to | | | | |
| | | 2303 | | | | |
| G4 | Female | 2401 | 3 | 2000 | 10 | Oral |
| | | to | | | | |
| | | 2403 | | | | |

Mortality, general symptoms, and changes in body weight were observed for 14 days after administration of the plasma radical solution, and as a result of examining the presence or absence of organ abnormalities, no dead animals were observed. In addition, as a result of observation of general symptoms and autopsy, abnormal findings due to administration of the plasma radical solution were not observed. From the results, the plasma radical solution was determined as Category 5 or Unclassified when classified by the Globally Harmonized Classification System (GHS) for Chemical Substances and Mixtures.

FIG. 18 is a diagram illustrating an evaluation result of a phototoxicity test of a plasma radical solution. The phototoxicity test was a test for evaluating a phototoxicity (PIF) induced by chemicals caused when exposed to light such as ultraviolet light, and the phototoxicity of the plasma radical solution to BALB/3T3 clone A31 cells was evaluated. Referring to FIG. 18, photo irritation factor (PIF) values and mean photo effect (MPE) values of the plasma radical solution and a positive control group (chlorpromazine hydrochloride, CPZ) can be confirmed. In the case of the positive control group, since the PIF value was measured as 18.882 and the MPE value was 0.418, phototoxicity was shown, whereas the plasma radical solution was measured as a PIF value of 1.000 and an MPE value of −0.008, and it may be confirmed that the plasma radical solution was a non-phototoxic substance.

As a result of various toxicity evaluations performed above, it can be confirmed that the plasma radical solution was a non-toxic material. Therefore, when the plasma radical solution was used on the human body, the plasma radical solution may be used in various bio-fields such as skin wound healing, acne relief, and atopy treatment.

<Method for Preparing Plasma Radical Solution>

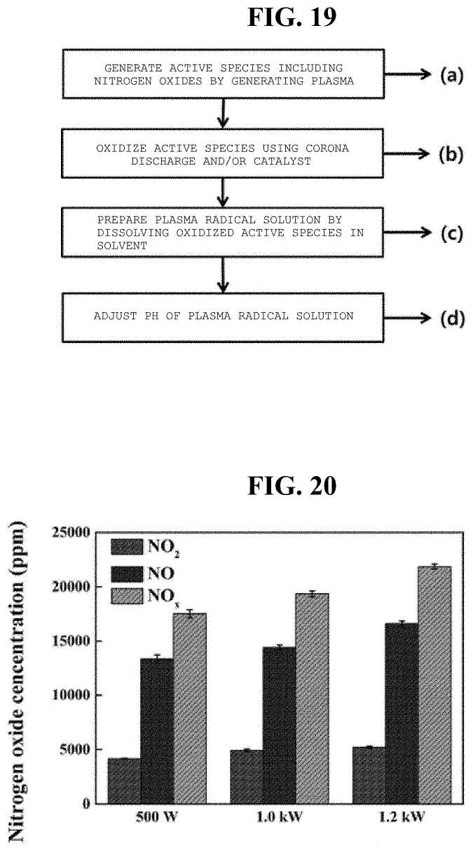

FIG. 19 is a flowchart of a method for preparing a plasma radical solution according to the present invention. The method for preparing the plasma radical solution according to the present invention may include Steps (a), (b), (c) and (d).

Step (a) according to the present invention may generate active species including nitrogen oxides by generating plasma. To generate plasma, a microwave plasma generating device was used. To generate the microwave plasma, 10 lpm of air at 500 W power, 15 lpm of air at 1.0 kW power, and 20 lpm of air at 1.2 kW power were injected, respectively, and as the plasma was generated, active species including nitrogen oxides may be generated. FIG. 20 is a graph showing the generation amounts of nitrogen oxides according to plasma power. Referring to FIG. 20, it can be seen that the amount of nitrogen oxides generated increased as higher power is supplied. It can be seen that nitrogen oxides generated at this time are mostly NO and NO₂.

Step (b) according to the present invention may oxidize the active species using at least one of a corona discharge and a catalyst. Among the nitrogen oxides generated in step (a), since the solubility of NO at room temperature was very low at about 56 mg/L, NO was difficult to be dissolved in liquid, and thus the NO needs to be oxidized to NO₂ and dissolved in a solvent. At this time, at least one of a corona discharge and a catalyst may be used to oxidize NO to NO₂. The active species oxidized to NO₂ may be hydrolyzed in a liquid, so that it is easy to be dissolved in a solvent, and NO₂ may be dissolved to generate a large amount of NO₂—, NO₃—, and ONOO—.

In step (c) according to the present invention, the plasma radical solution may be prepared by dissolving the oxidized active species in the solvent. At this time, the active species may be dissolved in the solvent by selecting any one of distilled water and purified water as the solvent. At this time, the active species may be dissolved in the solvent through bubbling. Since fine bubbles are formed in the solvent when the bubbling is performed, a reaction area between the active species and the solvent is increased, and thus, the active species may be better dissolved in the solvent.

Step (d) according to the present invention adjusts the pH of the prepared plasma radical solution to 0 to 4, so that the plasma radical solution may have bio-properties. At this time, NaH₂PO₄ may be used to adjust the pH. When the pH of the plasma radical solution is higher than 4, bio-properties may be reduced.

The present invention has been described with reference to the preferred embodiments. However, it will be appreciated by those skilled in the art that various modifications and changes of the present invention can be made without departing from the spirit and the scope of the present invention which are defined in the appended claims.

1. A plasma radical solution comprising active species that sterilize any one or more of bacteria, fungi and viruses; and a solvent in which the active species are dissolved,
wherein the active species are generated through plasma.

2. The plasma radical solution of claim 1, wherein the active species include at least one of reactive nitrogen species and reactive oxygen species, and
the reactive nitrogen species include peroxynitrite (ONOO—).

3. The plasma radical solution of claim 1, wherein the plasma radical solution sterilizes any one or more of bacteria, fungi and viruses at pH 0 to 4.

4. The plasma radical solution of claim 1, wherein the plasma radical solution sterilizes any one or more of bacteria, fungi and viruses at a NO₃— (nitrite) concentration of 400 ppm or higher.

5. The plasma radical solution of claim 1, wherein the plasma radical solution sterilizes any one or more of bacteria, fungi and viruses at conductivity of 1000 μs/cm or higher.

6. The plasma radical solution of claim 1, wherein the plasma radical solution is stored at a temperature of 85° C. or less.

7. The plasma radical solution of claim 1, wherein the plasma radical solution has an expiration period of 6 months or more.

8. The plasma radical solution of claim 1, wherein the plasma radical solution has a sterilization effect on the bacteria including any one or more of gram positive bacteria and gram negative bacteria,
wherein the gram positive bacteria include any one or more of Staphylococcus aureus, Bacillus cereus, Bacillus subtilis, Enterococcus, Streptococcus, and Lactococcus, and the gram negative bacteria include any one or more of Escherichia coli, Salmonella typhimurium, Pseudomonas aeruginosa, Klebsiella penumoniae, Haemophilus influenzae, Enterobacter aerogenes and Yersinia pestis.

9. The plasma radical solution of claim 1, wherein the plasma radical solution has a sterilization effect on the fungi including any one or more of Candida and Aspergillus.

10. The plasma radical solution of claim 1, wherein the plasma radical solution has a sterilization effect on the viruses including influenza virus.

11. The plasma radical solution of claim 1, wherein the plasma radical solution has bio-properties that are able to be used for antibacterial, skin wound healing, acne relief, atopy treatment, psoriasis treatment, dermatitis treatment, viral skin disease treatment, skin soothing and muscle regeneration.

12. The plasma radical solution of claim 1, wherein the solvent is selected by any one of distilled water and purified water.

13. A method for preparing a plasma radical solution comprising:
(a) generating active species including nitrogen oxides by generating plasma;
(b) oxidizing the active species using at least one of a corona discharge and a catalyst;
(c) preparing a plasma radical solution by dissolving the oxidized active species in a solvent; and
(d) adjusting the pH of the plasma radical solution.

14. The method for preparing the plasma radical solution of claim 13, wherein in the step (c), the oxidized active species are dissolved in the solvent through bubbling, and the plasma radical solution includes peroxynitrite (ONOO—).

15. The method for preparing the plasma radical solution of claim 13, wherein in step (d), the pH of the plasma radical solution is adjusted to 0 to 4.
